(19) United States
(12) Patent Application Publication
(10) Pub. No.: US 2021/0368901 A1
(43) Pub. Date: Dec. 2, 2021

(54) ANTIMICROBIAL WEARABLE ARTICLE

(71) Applicant: Biosafe Gloves LLC, Deer Park, NY (US)

(72) Inventors: Steven Richard Brown, Oyster Bay Cove, NY (US); Leonard Robert Butterman, Merrick, NY (US)

(21) Appl. No.: 17/329,660

(22) Filed: May 25, 2021

Related U.S. Application Data
(60) Provisional application No. 63/029,890, filed on May 26, 2020.

(51) Int. Cl. A41D 31/30; A41D 13/11; A41D 19/00; A61L 2/18; A61L 2/26; A01N 55/00

(52) U.S. Cl. CPC A41D 31/30; A41D 13/1192; A41D 19/0055; A61L 2202/26; A61L 2/26; A01N 55/00; A61L 2/18

(57) ABSTRACT

Disclosed herein are articles (e.g., wearable articles, such as gloves and masks) comprising an antimicrobial material, and methods of manufacture thereof. In various embodiments, the antimicrobial material comprises dimethyloctadecyl[3-(trimethoxysilyl)propyl]ammonium, or a salt thereof. In some embodiments, the antimicrobial article (e.g., a wearable article, such as a glove or mask) may have antiviral properties against coronavirus, such as SARS-CoV-2.

### BACKGROUND

### Field

The present application relates to the fields of chemistry, chemical engineering and medicine. More particularly, disclosed herein are articles with antimicrobial properties, as well as uses and methods of manufacture thereof.

### Description of the Related Art

In order to avoid contact with infectious microorganisms, articles, such as gloves and masks, may be utilized. However, the articles (e.g. gloves and masks) themselves may become carriers for infectious microorganism, and therefore once worn the gloves or masks may spread the microorganism to the wearer, other individuals and other surfaces. Furthermore, used gloves or masks may require disinfection before use again, or may require disposal altogether. Therefore, improved gloves and masks that avoid these drawbacks are of interest.

### SUMMARY

The following presents a simplified summary of one or more aspects in order to provide a basic understanding of such aspects. This summary is not an extensive overview of all contemplated aspects, and is intended to neither identify key or critical elements of all aspects nor delineate the scope of any or all aspects. Its sole purpose is to present some concepts of one or more aspects in a simplified form as a prelude to the more detailed description that is presented later.

In one aspect, a glove is provided. The glove includes an antimicrobial material, wherein the antimicrobial material comprises a microbiostatic agent.

In some embodiments, the microbiostatic agent is a quaternary ammonium organosilane compound, or a salt thereof. In some embodiments, the quaternary ammonium organosilane compound, or a salt thereof, is dimethyloctadecyl[3-(trimethoxysilyl)propyl]ammonium, or a salt thereof. In some embodiments, the dimethyloctadecyl[3-(trimethoxysilyl)propyl]ammonium, or a salt thereof, is dimethyloctadecyl[3-(trimethoxysilyl)propyl]ammonium chloride.

In some embodiments, the glove is configured to provide at least about 98% reduction of microbes. The glove of claim 1, wherein the glove is configured to provide at least about 99.5% reduction of microbes. In some embodiments, the glove is configured to provide at least about 99.9% reduction of microbes. In some embodiments, the glove comprises the microbiostatic agent at a concentration of about 20 μg/cm² to about 200 μg/cm².

In some embodiments, the glove is antimicrobial. In some embodiments, the glove is antimicrobial to microbes selected from the group consisting of bacteria, viruses, fungi, and combinations thereof. In some embodiments, the bacteria are selected from the group consisting of Staphylococcus aureus, Escherichia coli, Klebsiella spp., Streptococcus pneumonia, Listeria monocytogenes, Haemophilus influenzae, and combinations thereof. In some embodiments, the virus is a coronavirus. In some embodiments, the coronavirus is SARS-CoV-2.

In some embodiments, the glove further comprises a glove material selected from the group consisting of latex, rubber, vinyl, neoprene, cloth, wool, silk, and combinations thereof. In some embodiments, the rubber glove material is nitrile rubber. In some embodiments, the antimicrobial material is coated on an exterior surface of the glove. In some embodiments, the antimicrobial material is infused within the glove.

In another aspect, a method of forming an antimicrobial glove is provided. The method includes contacting a glove with an antimicrobial solution comprising a microbiostatic agent and a first solvent; and drying the glove to form an antimicrobial glove.

In some embodiments, the antimicrobial solution comprises the microbiostatic agent at a concentration of at most about 2 wt. %. In some embodiments, the antimicrobial solution comprises the microbiostatic agent at a concentration of about 0.2-0.6 wt. %. In some embodiments, the first solvent is selected from the group consisting of water, an alcohol, and combinations thereof.

In some embodiments, the method further comprises providing a concentrated antimicrobial solution comprising the microbiostatic agent and a second solvent; and adding the first solvent to the concentrated antimicrobial solution to form the antimicrobial solution. In some embodiments, the volume ratio of the concentrated antimicrobial solution to the first solvent is about 1:5 to about 1:20. In some embodiments, the second solvent is selected from the group consisting of water, an alcohol, and combinations thereof. In some embodiments, the first and second solvents are the same.

In some embodiments, contacting is selected from the group consisting of dipping the glove into the antimicrobial solution, spraying the glove with the antimicrobial solution, fogging the glove with the antimicrobial solution, and combinations thereof. In some embodiments, contacting comprises dipping the glove into the antimicrobial solution.

In another aspect, a method of forming an antimicrobial glove is provided. The method includes mixing a glove material with a microbiostatic agent to form a first solution; and forming the first solution into an antimicrobial glove.

In another aspect, a wearable article is provided. The wearable article includes an antimicrobial material, wherein the antimicrobial material comprises a microbiostatic agent.

In some embodiments, the microbiostatic agent is a quaternary ammonium organosilane compound, or a salt thereof. In some embodiments, the quaternary ammonium organosilane compound, or a salt thereof, is dimethyloctadecyl[3-(trimethoxysilyl)propyl]ammonium, or a salt thereof. In some embodiments, the dimethyloctadecyl[3-(trimethoxysilyl)propyl]ammonium, or a salt thereof, is dimethyloctadecyl[3-(trimethoxysilyl)propyl]ammonium chloride.

In some embodiments, the wearable article is configured to provide at least about 98% reduction of microbes. In some embodiments, the wearable article is configured to provide at least about 99.5% reduction of microbes. In some embodiments, the wearable article is configured to provide at least about 99.9% reduction of microbes.

The wearable article of claim 1, wherein the wearable article comprises the microbiostatic agent at a concentration of about 20 μg/cm² to about 200 μg/cm². In some embodiments, the wearable article is antimicrobial. In some embodiments, the wearable article is antimicrobial to microbes selected from the group consisting of bacteria, viruses, fungi, and combinations thereof. In some embodiments, the bacteria are selected from the group consisting of Staphylococcus aureus, Escherichia coli, Klebsiella spp., Streptococcus pneumonia, Listeria monocytogenes, Haemophilus influenzae, and combinations thereof. In some embodiments, the virus is a coronavirus. In some embodiments, the coronavirus is SARS-CoV-2.

In some embodiments, the wearable article is selected from the group consisting of a glove, a mask, and combinations thereof. In some embodiments, the wearable article is a glove. In some embodiments, the wearable article further comprising a glove material selected from the group consisting of latex, rubber, vinyl, neoprene, cloth, wool, silk, and combinations thereof. In some embodiments, the rubber glove material is nitrile rubber. In some embodiments, the wearable article is a mask. In some embodiments, the wearable article further comprising a mask material selected from the group consisting of cotton, polypropylene, fiberglass, polyester, a polyester blend, paper, latex, rubber (e.g., nitrile rubber), vinyl, neoprene, cloth, wool, leather, and silk, and combinations thereof. In some embodiments, the mask material comprises a filter material. In some embodiments, the mask is a multilayer mask.

In some embodiments, the antimicrobial material is coated on an exterior surface of the wearable article. In some embodiments, the antimicrobial material is infused within the wearable article.

In another aspect, a method of forming an antimicrobial wearable article is provided. The method includes: contacting a wearable article with an antimicrobial solution comprising a microbiostatic agent and a first solvent, and drying the contacted wearable article to form an antimicrobial wearable article.

In some embodiments, the antimicrobial solution comprises the microbiostatic agent at a concentration of at most about 2 wt. %. In some embodiments, the antimicrobial solution comprises the microbiostatic agent at a concentration of about 0.2-0.6 wt. %. In some embodiments, the first solvent is selected from the group consisting of water, an alcohol, and combinations thereof.

In some embodiments, the method further includes: providing a concentrated antimicrobial solution comprising the microbiostatic agent and a second solvent, and adding the first solvent to the concentrated antimicrobial solution to form the antimicrobial solution. In some embodiments, the volume ratio of the concentrated antimicrobial solution to the first solvent is about 1:5 to about 1:20. In some embodiments, the second solvent is selected from the group consisting of water, an alcohol, and combinations thereof. In some embodiments, the first and second solvents are the same.

In some embodiments, contacting is selected from the group consisting of dipping the wearable article into the antimicrobial solution, spraying the wearable article with the antimicrobial solution, fogging the wearable article with the antimicrobial solution, and combinations thereof. In some embodiments, contacting comprises dipping the wearable article into the antimicrobial solution.

In another aspect, a method of forming an antimicrobial wearable article is provided. The method includes: mixing an article material with a microbiostatic agent to form a first solution, and forming the first solution into an antimicrobial wearable article.

In another aspect, a method of forming an antimicrobial wearable article is provided. The method includes: providing at least one article material, contacting the article material with an antimicrobial solution comprising a microbiostatic agent and a first solvent, drying the article material, and processing the dried article material to form an antimicrobial wearable article.

In some embodiments, processing is selected from the group consisting of cutting, attaching a fastener, sewing, and combinations thereof.

### DETAILED DESCRIPTION

The present disclosure is related to articles (e.g. gloves and masks) comprising an antimicrobial material, and methods of fabricating thereof. The antimicrobial material comprises a microbiostatic agent that may have antimicrobial properties against microbes. For example, an antimicrobial glove or mask may have antimicrobial properties against Staphylococcus aureus and/or SARS-CoV-2.

As discussed herein, the article (e.g., a wearable article, such as a glove or mask) comprises an antimicrobial material. In some embodiments, the antimicrobial material comprises an antimicrobial agent selected from the group consisting of an antibiotic, an antibacterial, an antifungal, an antiviral, antiprotozoan, and combinations thereof. In some embodiments, the antimicrobial material comprises an antimicrobial agent selected from the group consisting of a microbiocidal agent, a microbiostatic agent, and combinations thereof. In some embodiments, the microbiostatic agent is a quaternary ammonium organosilane compound, or a salt thereof. For example, in some embodiments, the quaternary ammonium organosilane compound, or a salt thereof, is a quaternary ammonium organosilane free-base compound or a quaternary ammonium organosilane halide compound. In some embodiments, the quaternary ammonium organosilane salt compound has the structure of Formula (A), wherein Formula (A) has the structure:

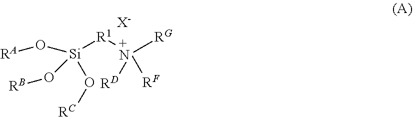

wherein each of R^{A}, R^{B}, R^{C}, R^{D}, R^{E}, R^{F} and R^{G} are individually selected form the group consisting of an optionally substituted alkyl, an optionally substituted alkenyl, and an optionally substituted alkynyl; wherein R¹ is selected from the group consisting of an optionally substituted alkylene, an optionally substituted alkenylene, and an optionally substituted alkynylene; and X⁻ is a halogen.

In some embodiments, the quaternary ammonium organosilane compound, or a salt thereof, is dimethyloctadecyl[3-(trimethoxysilyl)propyl]ammonium, or a salt thereof. For example, in some embodiments, the dimethyloctadecyl[3-(trimethoxysilyl)propyl]ammonium, or a salt thereof, is dimethyloctadecyl[3-(trimethoxysilyl)propyl]ammonium free-base or a dimethyloctadecyl[3-(trimethoxysilyl)propyl]ammonium halide. In some embodiments, the dimethyloctadecyl[3-(trimethoxysilyl)propyl]ammonium, or a salt thereof, is dimethyloctadecyl[3-(trimethoxysilyl)propyl]ammonium chloride (CAS No. 27668-52-6) (also named octadecylaminodimethyltrihydroxysilyl propyl ammonium chloride; 3-(trihydroxysilyl) propyldimethyloctadecyl ammonium chloride; or N,N-dimethyl-N-(3-(trimethoxysilyl)propyl)octadecan-1-aminium chloride), the structure of which is shown below:

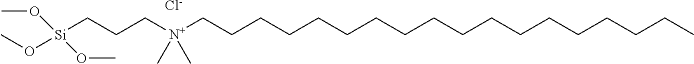 [SMILES: [Cl-] ; CCCCCCCCCCCCCCCCCC[N+](C)(C)CCC[Si](OC)(OC)OC]

In some embodiments, the article (e.g., a wearable article, such as a glove or mask) is configured to provide, provide about, provide at least, or provide at least about, a 90%, 95%, 98%, 99%, 99.5%, 99.8%, 99.9%, 99.95% or 99.99% reduction of microbes, or any range of values therebetween. In some embodiments, the article (e.g., a wearable article, such as a glove or mask) comprises a antimicrobial agent, for example a microbiostatic agent, at a concentration of, of about, of at least, or of at least about, 1 μg/cm², 5 μg/cm², 10 μg/cm², 20 μg/cm², 30 μg/cm², 50 μg/cm², 100 μg/cm², 150 μg/cm², 200 μg/cm², 250 μg/cm², 300 μg/cm², 500 μg/cm² or 1000 μg/cm², or any range of values therebetween, based on the surface area of the article (e.g., a wearable article, such as a glove or mask).

In some embodiments, the article (e.g., a wearable article, such as a glove or mask) is antimicrobial. In some embodiments, the article (e.g., a wearable article, such as a glove or mask) is antimicrobial to microbes selected from the group consisting of bacteria, viruses, fungi, and combinations thereof. Non-limiting examples of bacteria include Staphylococcus aureus, Escherichia coli, Klebsiella spp., Streptococcus pneumonia, Listeria monocytogenes and Haemophilus influenzae. Non-limiting examples of viruses include a coronavirus, for example such as SARS-CoV-2. In some embodiments, the article (e.g., a wearable article, such as a glove or mask) is configured to retain antimicrobial properties for, for about, for at least, or for at least about, 1 day, 7 days, 14 days, 30 days, 60 days, 90 days, 120 days, 150 days, 180 days, 210 days, 240 days, 270 days, 300 days, 330 days, 1 year, 1.5 years, 2 years or 3 years, or any range of values therebetween.

The glove may comprise any suitable glove material, and combinations thereof. Non-limiting examples of glove materials include latex, rubber (e.g., nitrile rubber), vinyl, neoprene, cloth, wool, leather, and silk. In some embodiments, the antimicrobial material is coated on an exterior surface of the glove. In some embodiments, the antimicrobial material is infused within the glove.

The mask may comprise any suitable mask material, and combinations thereof. In some embodiments, the mask material comprises a fabric material. Non-limiting examples of mask materials include cotton, polypropylene, fiberglass, polyester, a polyester blend, paper, latex, rubber (e.g., nitrile rubber), vinyl, neoprene, cloth, wool, leather, and silk. In some embodiments, the mask material includes a filter material, such as a high efficiency particulate air (HEPA) material. In some embodiments, the mask is a multilayer mask. In some embodiments, the antimicrobial material is coated on one or more surfaces of the mask (e.g. an exterior, intermediary or interior layer surface). In some embodiments, the antimicrobial material is infused within the mask.

As noted above, the antimicrobial article (e.g., a wearable article, such as a glove or mask) may be formed by coating or infusing an antimicrobial agent to an article (e.g., a wearable article, such as a glove or mask). In some embodiments, the antimicrobial article (e.g., a wearable article, such as a glove or mask) is prepared by contacting an article (e.g., a wearable article, such as a glove or mask) with an antimicrobial solution comprising an antimicrobial agent (e.g., a microbiostatic agent) and a first solvent, and then drying the article (e.g., a wearable article, such as a glove or mask) to form the antimicrobial article (e.g., a wearable article, such as a glove or mask). In some embodiments, the antimicrobial agent may be coated on an exterior surface of the article (e.g., a wearable article, such as a glove or mask). In some embodiments, the coated antimicrobial agent may infuse into the article (e.g., a wearable article, such as a glove or mask) material. In some embodiments, the first solvent is selected from the group consisting of water, an alcohol, and combinations thereof.

In order to adhere the antimicrobial solution to the article (e.g., a wearable article, such as a glove or mask), it was surprisingly discovered that high concentrations of the antimicrobial agent in the antimicrobial solution provided poor coating of the article (e.g., a wearable article, such as a glove or mask), and that unexpectedly lower concentrations of the antimicrobial agent in the antimicrobial solution provided improved coatings on the article (e.g., a wearable article, such as a glove or mask). In some embodiments, the antimicrobial solution comprises antimicrobial agent (e.g., the microbiostatic agent) at a concentration of, of about, of at most, or of at most about, 5 wt. %, 4 wt. %, 3 wt. %, 2 wt. %, 1 wt. %, 0.9 wt. %, 0.8 wt. %, 0.7 wt. %, 0.6 wt. %, 0.5 wt. %, 0.4 wt. %, 0.3 wt. %, 0.2 wt. %, 0.1 wt. % or 0.05 wt. %, or any range of values therebetween.

In order to achieve sufficiently low concentrations of the antimicrobial agent in the antimicrobial solution, a concentrated antimicrobial solution may be diluted. In some embodiments, a concentrated antimicrobial solution comprises the microbiostatic agent and a second solvent, and the first solvent is added to the concentrated antimicrobial solution to form the antimicrobial solution. In some embodiments, the volume ratio of the concentrated antimicrobial solution to the first solvent is, or is about, 1:5, 1:6, 1:7, 1:8, 1:9, 1:10, 1:12, 1:14, 1:16, 1:18 or 1:20, or any range of values therebetween. In some embodiments, the concentrated antimicrobial solution comprises antimicrobial agent (e.g., the microbiostatic agent) at a concentration of, of about, of at least, or of at least about, 2 wt. %, 3 wt. %, 4 wt. %, 5 wt. %, 6 wt. %, 8 wt. % or 10 wt. %, or any range of values therebetween. In some embodiments, the second solvent is selected from the group consisting of water, an alcohol, and combinations thereof. In some embodiments, the first and second solvents are the same. In some embodiments, the first and second solvents are different solvents.

The article (e.g., a wearable article, such as a glove or mask) may be contacted with the antimicrobial solution by a variety of methods. In some embodiments, contacting performed by dipping the article (e.g., a wearable article, such as a glove or mask) into the antimicrobial solution, soaking the article (e.g., a wearable article, such as a glove or mask) into the antimicrobial solution, spraying the article (e.g., a wearable article, such as a glove or mask) with the antimicrobial solution, and/or fogging the article (e.g., a wearable article, such as a glove or mask) with the antimicrobial solution. In some embodiments, dipping or soaking further comprises agitation of the article in the antimicrobial solution. In some embodiments, agitation includes spinning, tumbling and/or mixing of the article and/or the antimicrobial solution.

In some embodiments, the article (e.g., a wearable article, such as a glove or mask) is placed into a treatment-drying apparatus, treated with the antimicrobial solution, and dried using a heater to form the antimicrobial article. In some embodiments, the treatment is performed by dipping, soaking, spraying and/or fogging with the antimicrobial solution. In some embodiments, the article is a glove.

In some embodiments, the article (e.g., a wearable article, such as a glove or mask) and/or at least one article material is placed on a conveyer, treated with the antimicrobial solution, and dried using a heater. In some embodiments, the treatment is performed by dipping, soaking, spraying and/or fogging with the antimicrobial solution. In some embodiments, at least one article material is provided on a material roll, and unrolled prior to treatment. In some embodiments, the at least one article material is further processed subsequent to drying to form the antimicrobial article. In some embodiments, further processing may include at least one of cutting, attaching a fastener (e.g. ear loops) and sewing. In some embodiments, the article is a mask.

Alternatively, or in addition, the antimicrobial article (e.g., a wearable article, such as a glove or mask) may be prepared by forming an article (e.g., a wearable article, such as a glove or mask) from a solution comprising the antimicrobial agent (e.g., the microbiostatic agent). In some embodiments, the antimicrobial article (e.g., a wearable article, such as a glove or mask) is prepared by mixing an article (e.g., a wearable article, such as a glove or mask) material with a microbiostatic agent to form a first solution, and forming the first solution into an antimicrobial article (e.g., a wearable article, such as a glove or mask). In some embodiments, forming comprises dipping and/or soaking an article (e.g., a wearable article, such as a glove or mask) into the first solution (e.g., for a specified period of time) to form an adhered article (e.g., a wearable article, such as a glove or mask) coating. In some embodiments, forming comprises drying the adhered article (e.g., a wearable article, such as a glove or mask) coating (e.g., at specified temperature or temperature range, for a specified period of time) to form the antimicrobial article (e.g., a wearable article, such as a glove or mask).

### EXAMPLES

### Objective

An example test study verifying the efficacy of certain disclosed embodiments will now be described. The objective of this study was to evaluate the bactericidal activity of the test samples. The test procedure was to simulate the way in which the glove is intended to be used.

### Summary of Results

The test substances are Glove Materials A, E, I and J, wherein Glove Material E is the Control Glove Material and Glove Materials A, I and J are the Treated Glove Materials treated with BioProtect™ 500, which contains 5.0 wt % dimethyloctadecyl[3-(trimethoxysilyl)propyl]ammonium chloride (ViaClean Technologies LLC). Glove Materials A, E, I and J are nitrile glove materials. Treated Glove Materials A, I and J were prepared by dipping and coating the Treated Glove Materials in a diluted solution of BioProtect™ 500, and then drying the Treated Glove Materials. The diluted solutions of BioProtect™ 500 contained 0.25 wt. % microbiostatic agent for Treated Glove Material A, 0.5 wt. % microbiostatic agent (not leached) for Treated Glove Material I, and 0.5 wt. % microbiostatic agent for Treated Glove Material J.

Three 2 cm diameter portions of each of the Glove Materials were used for testing.

The test organism Staphylococcus aureus (ATCC #6538) was provided to the Glove Materials with an exposure time of 120 minutes at room temperature (22° C.±2° C.). Letheen Broth was used as a neutralizer. A 5% (final concentration) of fetal bovine serum (FBS) was added to the microbial test suspension as the soil load.

Efficacy Result: Glove Material samples A, I and J demonstrated 2.69, 3.95 and 3.18 log 10 reductions in bacterial viability, respectively.

### Test System

Staphylococcus aureus (ATCC #6538) was used as the microbial challenge. The strain was obtained directly by CREM Co Labs from the American Type Culture Collection (ATCC), Manassas, Va. It is a Gram-positive coccus frequently incriminated in healthcare-associated infections.

The growth medium used in this study was Trypticase soy broth (TSB) and Trypticase soy agar (TSA) as the medium for the organism's recovery from the test and control samples.

To prepare a broth culture, a 100-μL volume of the stock culture of the test organism was added to 10.0 mL of TSB in a tube and incubated aerobically for 20±2 h at 36±1° C. without shaking.

The Test Inocula was prepared and tested with an Efficacy Test as follows: First, the bacterial broth culture was diluted 10-fold by adding 100 μL of an overnight culture in 900 μL of PBS (pH 7.2±2). The soil load was then added to it.

### Test Methods

To prepare the test substances, the Glove Materials A, E, I and J were cut into 2-centimeter diameter disks and were sterilized using UV box twice with 5 minutes exposure times.

The Efficacy Test was performed as follows: A 20 μL volume of the microbial test suspension with the soil load was placed on each carrier as 20×1 uL droplets using a calibrated positive-displacement pipette with a 10-μL tip. The contact time was calculated from the time of putting the bacterial inoculum on the carrier using a calibrated timer. After the exposure time, three carriers of each group were removed from the Petri dish and each carrier was separately placed into a Nalgene vial containing 10.0 mL of the neutralizer/eluent/diluent (Letheen Broth) and vortex mixed for 30±5 seconds to recover the inocula from the carriers (100 dilution).

A 10-fold dilution series was prepared for each test and control using PBS. Depending on the initial inoculum level and the level of microbicidal activity expected, the number of dilutions was the same in test and control (five dilutions). All dilutions of eluates treated carriers were membrane-filtered using a vacuum, and then the carrier vial was rinsed four times with 15 mL of PBS. The membranes were washed with 10 mL PBS before pouring the contents of each vial and washed with 40 mL of PBS after pouring the contents of each vial. Finally, each membrane was plated aseptically on the surface of a TSA plate.

The plates were incubated aerobically at 36±1° C. for 48±4 hours and the colony-forming units (CFU) of the test organism on each plate were counted. The plates were re-incubated for another three days and examined again to detect the presence of any late-growing colonies due to stressed or injured organisms.

Purity of the test organism: A vial with lyophilized powder of the test organism (S. aureus; ATCC #6538) was purchased by CREM Co Labs directly from ATCC. The culture was aseptically rehydrated in 5 mL of TSB at CREM Co Labs as instructed by the supplier. The resulting suspension was aliquoted as 500 μL volumes into sterile 2-mL plastic cryovials for storage at −80° C. Each vial was labelled with the name of the organism, date of preparation of the suspension and the passage # as ‘0’.

In order to confirm the purity of the culture, a series (10-1 to 10-4) of 10-fold dilutions of the original culture suspension was prepared using sterile TSB as the diluent. A 100 μL volume from undiluted suspension as well as from dilutions 10-2 and 10-4 was separately spread on Petri plates (100 mm diameter) containing TSA. The plates were incubated aerobically at 36±1° C. for 22±2 hours.

Sterility Controls: Controls were run to check the sterility of the carriers, neutralizer, PBS, PBST, Letheen Broth, culture broth (TSB) and agar (TSA) as follows:

For the TSA, two plates from each lot and for TSB 10 mL of each lot were incubated and held at 36±1° C. for a minimum of 5 days.

Two carriers from each set were placed in a tube with sterile Letheen Broth and incubated at 36±1° C. held for at least 5 days.

A 2% of volume from each reagent (neutralizer, diluent components was passed separately through a membrane filter (0.22 μm pore diam.) and each membrane individually placed on a TSA plate (100 mm) and incubated at 36±1° C. for at least 5 days.

Growth Control: The culture media (TSA, TSB, and Letheen Broth) used in this study were tested for their ability to support the growth S. aureus by inoculation of a plate or tube followed by incubation at 36±1° C.

The efficacy of the Treated Glove Materials were assessed as follows: 3 carriers for each test sample and three negative controls were utilized. The test was initiated with the first control carrier following 5 test carriers, then second control carrier following 4 other test carriers and ended up with the third control carrier to capture the natural decay of the bacteria.

The initial titer of the stock culture of S. aureus was estimated by making serial dilutions using TSB. In total, 8 10-fold dilutions were prepared and the last three dilutions (10-6, 10-7 and 10-8) were placed on TSA plates and incubated at 36±1° C. for 22±2 hrs.

### Data Analysis

The calculation of a Log₁₀ reduction is shown below:

Log₁₀ Reduction=[Log₁₀ of average CFU recovered from control carriers]−[Log₁₀ of average CFU recovered from the test carriers]

The calculation of % reduction is shown below:

% Reduction=([Average CFU recovered from control carriers]−[Average CFU recovered from the test carriers])×100/[Average CFU recovered from control carriers]

### Study Acceptance Criteria

Culture Purity Control: For a valid test, the test culture was required to show a pure culture of S. aureus with its typical morphology and colony color and with no evidence for contamination. All sterility controls were also to be negative for a valid test. There was to be readily visible growth of the test organism in all the inoculated culture media for the test to be considered as valid. The test formulation was to show a ≥3 log₁₀ in the viability titer of the test organism in the tested contact time to meet the product performance criterion.

### Test Results

Culture Purity Control: The original culture suspension was used to check for culture purity. The growth obtained showed typical colony morphology (moist round colonies with a 2-3 mm diameter) and color (golden) for S. aureus.

Sterility Controls: All sterility controls were found to be negative.

Initial Titer of Bacterial Suspension: The initial titer of the stock culture was 108-109 CFU/mL

Efficacy Test: The numbers of CFU in each dilution of control (sample E) and test substances A, I and J for Test #1 and Test #2 are summarized in Tables 1 and 2, respectively. Test #1 and #2 are performed under these same testing protocols described herein. The log₁₀ reductions and percentage reductions of each sample were calculated using a validated Excel sheet and summarized in Table 3. Samples A, I and J demonstrate 2.69, 3.95 and 3.18 log₁₀ reduction, respectively (99.74, 99.97 and 99.93 percent reduction) in the CFU for bactericidal activity.

TABLE 1
CFU in each dilution of control (Sample E) and
test substances (A, I and J) in Test #1

| | Dilution | | | | | | |
|---|---|---|---|---|---|---|---|
| | 10⁰ | 10⁻¹ | 10⁻² | 10⁻³ | 10⁻⁴ | 10⁻⁵ | 10⁻⁶ |
| E1 | TNTC | TNTC | TNTC | 26 | 2 | 0 | 0 |
| E2 | TNTC | TNTC | TNTC | 20 | 4 | 0 | 0 |
| E3 | TNTC | TNTC | TNTC | 58 | 6 | 1 | 0 |
| I1 | TNTC | 35 | 3 | 1 | 0 | 0 | 0 |
| I2 | TNTC | 16 | 1 | 0 | 0 | 0 | 0 |
| I3 | 0 | 26 | 6 | 0 | 0 | 0 | 0 |
| A1 | 12 | 0 | 0 | 0 | 0 | 0 | 0 |
| A2 | 2 | 0 | 0 | 0 | 0 | 0 | 0 |
| A3 | 7 | 1 | 0 | 0 | 0 | 0 | 0 |

TNTC: Too Numerous to Count;
NT: Not tested

TABLE 2
CFU in each dilution of control (Sample E) and
test substances (A, I and J) in Test #2

| | Dilution | | | | | | |
|---|---|---|---|---|---|---|---|
| | 10⁰ | 10⁻¹ | 10⁻² | 10⁻³ | 10⁻⁴ | 10⁻⁵ | 10⁻⁶ |
| E1 | TNTC | TNTC | TNTC | TNTC | 48 | 9 | 2 |
| E2 | TNTC | TNTC | TNTC | TNTC | 43 | 5 | 1 |
| E3 | TNTC | TNTC | TNTC | TNTC | 51 | 7 | 1 |
| I1 | TNTC | 75 | 11 | 3 | 0 | 0 | 0 |
| I2 | TNTC | 79 | 10 | 3 | 0 | 0 | 0 |
| I3 | TNTC | 73 | 7 | 3 | 0 | 0 | 0 |
| A1 | TNTC | 59 | 8 | 0 | 0 | 0 | 0 |
| A2 | TNTC | 73 | 8 | 0 | 0 | 0 | 0 |
| A3 | TNTC | 115 | 19 | 1 | 0 | 0 | 0 |
| J1 | TNTC | 104 | 13 | 1 | 0 | 0 | 0 |
| J2 | TNTC | 97 | 21 | 1 | 0 | 0 | 0 |
| J3 | TNTC | 90 | 11 | 0 | 0 | 0 | 0 |

TNTC: Too Numerous to Count;
NT: Not tested

TABLE 3
Log₁₀ reductions and % reductions of the
three treated glove samples by the carrier
test method using S. aureus, sample E was considered as control.

| | Log₁₀ Reduction | | | % Reduction | | |
|---|---|---|---|---|---|---|
| Sample ID | Test #1 | Test #2 | Average | Test #1 | Test #2 | Average |
| Sample I | 2.37 | 3.01 | 2.69 | 99.57 | 99.90 | 99.74 |
| Sample A | 4.6 | 3.30 | 3.95 | 99.997 | 99.95 | 99.97 |
| Sample J | NT | 3.18 | 3.18 | NT | 99.93 | 99.93 |

TNTC: Too Numerous to Count;
NT: Not tested

### Definitions

Unless defined otherwise, all technical and scientific terms used herein have the same meaning as is commonly understood by one of ordinary skill in the art. All patents, applications, published applications and other publications referenced herein are incorporated by reference in their entirety unless stated otherwise. In the event that there are a plurality of definitions for a term herein, those in this section prevail unless stated otherwise.

Whenever a group is described as being “optionally substituted” that group may be unsubstituted or substituted with one or more of the indicated substituents. Likewise, when a group is described as being “unsubstituted or substituted” if substituted, the substituent(s) may be selected from one or more the indicated substituents. If no substituents are indicated, it is meant that the indicated “optionally substituted” or “substituted” group may be substituted with one or more group(s) individually and independently selected from alkyl, alkenyl, alkynyl, cycloalkyl, cycloalkenyl, aryl, heteroaryl, heterocyclyl, aryl(alkyl), cycloalkyl(alkyl), heteroaryl(alkyl), heterocyclyl(alkyl), hydroxy, alkoxy, acyl, cyano, halogen, thiocarbonyl, O-carbamyl, N-carbamyl, O-thiocarbamyl, N-thiocarbamyl, C-amido, N-amido, S-sulfonamido, N-sulfonamido, C-carboxy, O-carboxy, nitro, sulfenyl, sulfinyl, sulfonyl, haloalkyl, haloalkoxy, an amino, a mono-substituted amine group, a di-substituted amine group, a mono-substituted amine(alkyl) and a di-substituted amine(alkyl).

As used herein, “Cₐ to C_{b}” in which “a” and “b” are integers refer to the number of carbon atoms in a group. The indicated group can contain from “a” to “b”, inclusive, carbon atoms. Thus, for example, a “C₁ to C₄ alkyl” group refers to all alkyl groups having from 1 to 4 carbons, that is, CH₃—, CH₃CH₂—, CH₃CH₂CH₂—, (CH₃)₂CH—, CH₃CH₂CH₂CH₂—, CH₃CH₂CH(CH₃)— and (CH₃)₃C—. If no “a” and “b” are designated, the broadest range described in these definitions is to be assumed.

As used herein, the term “alkyl” refers to a fully saturated aliphatic hydrocarbon group. The alkyl moiety may be branched or straight chain. Examples of branched alkyl groups include, but are not limited to, iso-propyl, sec-butyl, t-butyl and the like. Examples of straight chain alkyl groups include, but are not limited to, methyl, ethyl, n-propyl, n-butyl, n-pentyl, n-hexyl, n-heptyl and the like. The alkyl group may have 1 to 30 carbon atoms (whenever it appears herein, a numerical range such as “1 to 30” refers to each integer in the given range; e.g., “1 to 30 carbon atoms” means that the alkyl group may consist of 1 carbon atom, 2 carbon atoms, 3 carbon atoms, etc., up to and including 30 carbon atoms, although the present definition also covers the occurrence of the term “alkyl” where no numerical range is designated). The alkyl group may also be a medium size alkyl having 1 to 12 carbon atoms. The alkyl group could also be a lower alkyl having 1 to 6 carbon atoms. An alkyl group may be substituted or unsubstituted.

As used herein, the term “alkylene” refers to a bivalent fully saturated straight chain aliphatic hydrocarbon group. Examples of alkylene groups include, but are not limited to, methylene, ethylene, propylene, butylene, pentylene, hexylene, heptylene and octylene. An alkylene group may be represented by [symbol] , followed by the number of carbon atoms, followed by a “*”. For example,

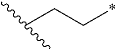 [SMILES: *CC*]

to represent ethylene. The alkylene group may have 1 to 30 carbon atoms (whenever it appears herein, a numerical range such as “1 to 30” refers to each integer in the given range; e.g., “1 to 30 carbon atoms” means that the alkyl group may consist of 1 carbon atom, 2 carbon atoms, 3 carbon atoms, etc., up to and including 30 carbon atoms, although the present definition also covers the occurrence of the term “alkylene” where no numerical range is designated). The alkylene group may also be a medium size alkyl having 1 to 12 carbon atoms. The alkylene group could also be a lower alkyl having 1 to 4 carbon atoms. An alkylene group may be substituted or unsubstituted. For example, a lower alkylene group can be substituted by replacing one or more hydrogen of the lower alkylene group and/or by substituting both hydrogens on the same carbon with a C₃₋₆ monocyclic cycloalkyl group (e.g.,

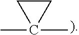

The term “alkenyl” used herein refers to a monovalent straight or branched chain radical of from two to twenty carbon atoms containing a carbon double bond(s) including, but not limited to, 1-propenyl, 2-propenyl, 2-methyl-1-propenyl, 1-butenyl, 2-butenyl and the like. An alkenyl group may be unsubstituted or substituted.

The term “alkynyl” used herein refers to a monovalent straight or branched chain radical of from two to twenty carbon atoms containing a carbon triple bond(s) including, but not limited to, 1-propynyl, 1-butynyl, 2-butynyl and the like. An alkynyl group may be unsubstituted or substituted.

The term “halogen atom” or “halogen” as used herein, means any one of the radio-stable atoms of column 7 of the Periodic Table of the Elements, such as, fluorine, chlorine, bromine and iodine.

While certain embodiments of the inventions have been described, these embodiments have been presented by way of example only, and are not intended to limit the scope of the disclosure. Indeed, the novel methods and systems described herein may be embodied in a variety of other forms. Furthermore, various omissions, substitutions and changes in the systems and methods described herein may be made without departing from the spirit of the disclosure. The accompanying claims and their equivalents are intended to cover such forms or modifications as would fall within the scope and spirit of the disclosure. Accordingly, the scope of the present inventions is defined only by reference to the appended claims.

Features, materials, characteristics, or groups described in conjunction with a particular aspect, embodiment, or example are to be understood to be applicable to any other aspect, embodiment or example described in this section or elsewhere in this specification unless incompatible therewith. All of the features disclosed in this specification (including any accompanying claims, abstract and drawings), and/or all of the steps of any method or process so disclosed, may be combined in any combination, except combinations where at least some of such features and/or steps are mutually exclusive. The protection is not restricted to the details of any foregoing embodiments. The protection extends to any novel one, or any novel combination, of the features disclosed in this specification (including any accompanying claims, abstract and drawings), or to any novel one, or any novel combination, of the steps of any method or process so disclosed.

Furthermore, certain features that are described in this disclosure in the context of separate implementations can also be implemented in combination in a single implementation. Conversely, various features that are described in the context of a single implementation can also be implemented in multiple implementations separately or in any suitable subcombination. Moreover, although features may be described above as acting in certain combinations, one or more features from a claimed combination can, in some cases, be excised from the combination, and the combination may be claimed as a subcombination or variation of a subcombination.

Moreover, while operations may be depicted in the drawings or described in the specification in a particular order, such operations need not be performed in the particular order shown or in sequential order, or that all operations be performed, to achieve desirable results. Other operations that are not depicted or described can be incorporated in the example methods and processes. For example, one or more additional operations can be performed before, after, simultaneously, or between any of the described operations. Further, the operations may be rearranged or reordered in other implementations. Those skilled in the art will appreciate that in some embodiments, the actual steps taken in the processes illustrated and/or disclosed may differ from those shown in the figures. Depending on the embodiment, certain of the steps described above may be removed, others may be added. Furthermore, the features and attributes of the specific embodiments disclosed above may be combined in different ways to form additional embodiments, all of which fall within the scope of the present disclosure. Also, the separation of various system components in the implementations described above should not be understood as requiring such separation in all implementations, and it should be understood that the described components and systems can generally be integrated together in a single product or packaged into multiple products. For example, any of the components for an energy storage system described herein can be provided separately, or integrated together (e.g., packaged together, or attached together) to form an energy storage system.

For purposes of this disclosure, certain aspects, advantages, and novel features are described herein. Not necessarily all such advantages may be achieved in accordance with any particular embodiment. Thus, for example, those skilled in the art will recognize that the disclosure may be embodied or carried out in a manner that achieves one advantage or a group of advantages as taught herein without necessarily achieving other advantages as may be taught or suggested herein.

Conditional language, such as “can,” “could,” “might,” or “may,” unless specifically stated otherwise, or otherwise understood within the context as used, is generally intended to convey that certain embodiments include, while other embodiments do not include, certain features, elements, and/or steps. Thus, such conditional language is not generally intended to imply that features, elements, and/or steps are in any way required for one or more embodiments or that one or more embodiments necessarily include logic for deciding, with or without user input or prompting, whether these features, elements, and/or steps are included or are to be performed in any particular embodiment.

Conjunctive language such as the phrase “at least one of X, Y, and Z,” unless specifically stated otherwise, is otherwise understood with the context as used in general to convey that an item, term, etc. may be either X, Y, or Z. Thus, such conjunctive language is not generally intended to imply that certain embodiments require the presence of at least one of X, at least one of Y, and at least one of Z.

Language of degree used herein, such as the terms “approximately,” “about,” “generally,” and “substantially” as used herein represent a value, amount, or characteristic close to the stated value, amount, or characteristic that still performs a desired function or achieves a desired result.

The scope of the present disclosure is not intended to be limited by the specific disclosures of preferred embodiments in this section or elsewhere in this specification, and may be defined by claims as presented in this section or elsewhere in this specification or as presented in the future. The language of the claims is to be interpreted broadly based on the language employed in the claims and not limited to the examples described in the present specification or during the prosecution of the application, which examples are to be construed as non-exclusive.

What is claimed is:

1. A wearable article comprising an antimicrobial material, wherein the antimicrobial material comprises a microbiostatic agent.

2. The wearable article of claim 1, wherein the microbiostatic agent is a quaternary ammonium organosilane compound, or a salt thereof.

3. The wearable article of claim 2, wherein the quaternary ammonium organosilane compound, or a salt thereof, is dimethyloctadecyl[3-(trimethoxysilyl)propyl]ammonium, or a salt thereof.

4. The wearable article of claim 3, wherein the dimethyloctadecyl[3-(trimethoxysilyl)propyl]ammonium, or a salt thereof, is dimethyloctadecyl[3-(trimethoxysilyl)propyl]ammonium chloride.

5. The wearable article of claim 1, wherein the wearable article is configured to provide at least about 98% reduction of microbes.

6. The wearable article of claim 1, wherein the wearable article is configured to provide at least about 99.5% reduction of microbes.

7. The wearable article of claim 1, wherein the wearable article is configured to provide at least about 99.9% reduction of microbes.

8. The wearable article of claim 1, wherein the wearable article comprises the microbiostatic agent at a concentration of about 20 μg/cm² to about 200 μg/cm².

9. The wearable article of claim 1, wherein the wearable article is antimicrobial.

10. The wearable article of claim 9, wherein the wearable article is antimicrobial to microbes selected from the group consisting of bacteria, viruses, fungi, and combinations thereof.

11. The wearable article of claim 10, wherein the bacteria are selected from the group consisting of Staphylococcus aureus, Escherichia coli, Klebsiella spp., Streptococcus pneumonia, Listeria monocytogenes, Haemophilus influenzae, and combinations thereof.

12. The wearable article of claim 10, wherein the virus is a coronavirus.

13. The wearable article of claim 12, wherein the coronavirus is SARS-CoV-2.

14. The wearable article of claim 1, wherein the wearable article is selected from the group consisting of a glove, a mask, and combinations thereof.

15. The wearable article of claim 14, wherein the wearable article is a glove.

16. The wearable article of claim 15, further comprising a glove material selected from the group consisting of latex, rubber, vinyl, neoprene, cloth, wool, silk, and combinations thereof.

17. The wearable article of claim 16, wherein the rubber glove material is nitrile rubber.

18. The wearable article of claim 14, wherein the wearable article is a mask.

19. The wearable article of claim 18, further comprising a mask material selected from the group consisting of cotton, polypropylene, fiberglass, polyester, a polyester blend, paper, latex, rubber (e.g., nitrile rubber), vinyl, neoprene, cloth, wool, leather, and silk, and combinations thereof.

20. The wearable article of claim 19, wherein the mask material comprises a filter material.

21. The wearable article of claim 18, wherein the mask is a multilayer mask.

22. The wearable article of claim 1, wherein the antimicrobial material is coated on an exterior surface of the wearable article.

23. The wearable article of claim 1, wherein the antimicrobial material is infused within the wearable article.

24. A method of forming an antimicrobial wearable article, comprising:
contacting a wearable article with an antimicrobial solution comprising a microbiostatic agent and a first solvent; and
drying the contacted wearable article to form an antimicrobial wearable article.

25. The method of claim 24, wherein the antimicrobial solution comprises the microbiostatic agent at a concentration of at most about 2 wt. %.

26. The method of claim 24, wherein the antimicrobial solution comprises the microbiostatic agent at a concentration of about 0.2-0.6 wt. %.

27. The method of claim 24, wherein the first solvent is selected from the group consisting of water, an alcohol, and combinations thereof.

28. The method of claim 24, further comprising:
providing a concentrated antimicrobial solution comprising the microbiostatic agent and a second solvent; and
adding the first solvent to the concentrated antimicrobial solution to form the antimicrobial solution.

29. The method of claim 28, wherein the volume ratio of the concentrated antimicrobial solution to the first solvent is about 1:5 to about 1:20.

30. The method of claim 28, wherein the second solvent is selected from the group consisting of water, an alcohol, and combinations thereof.

31. The method of claim 28, wherein the first and second solvents are the same.

32. The method of claim 24, wherein contacting is selected from the group consisting of dipping the wearable article into the antimicrobial solution, spraying the wearable article with the antimicrobial solution, fogging the wearable article with the antimicrobial solution, and combinations thereof.

33. The method of claim 32, wherein contacting comprises dipping the wearable article into the antimicrobial solution.

34. A method of forming an antimicrobial wearable article, comprising:
mixing an article material with a microbiostatic agent to form a first solution; and
forming the first solution into an antimicrobial wearable article.

35. A method of forming an antimicrobial wearable article, comprising:
providing at least one article material;
contacting the article material with an antimicrobial solution comprising a microbiostatic agent and a first solvent;
drying the article material; and
processing the dried article material to form an antimicrobial wearable article.

36. The method of claim 35, wherein processing is selected from the group consisting of cutting, attaching a fastener, sewing, and combinations thereof.
